(19) United States
(12) Patent Application Publication
(10) Pub. No.: US 2022/0273698 A1
(43) Pub. Date: Sep. 1, 2022

(54) MEDICINAL AND/OR PHARMACEUTICAL COMPOSITIONS FOR INTRAVESICAL INSTILLATION, PREPARATION AND USE THEREOF

(71) Applicants: Gabor RENY, Budapest (HU); Sandor LOVASZ, Budapest (HU); Janos GIBER, Budapest (HU); Peter BIRINYI, Budapest (HU)

(72) Inventors: Sandor LOVASZ, Budapest (HU); Gábor RÉNYI, Budapest (HU); János GIBER, Budapest (HU); Péter BIRINYI, Budapest (HU)

(21) Appl. No.: 17/628,112

(22) PCT Filed: Sep. 15, 2020
(86) PCT No.: PCT/HU2020/000026

§ 371 Date: Jan. 18, 2022

(30) Foreign Application Priority Data
Jul. 18, 2019 (HU) P1900257
Mar. 12, 2020 (HU) P2000094

(51) Int. Cl. A61K 31/727; A61K 31/728; A61K 31/737; A61K 31/167; A61K 31/196; A61K 31/573; A61K 47/02; A61K 9/107

(52) U.S. Cl. CPC A61K 31/727; A61K 31/728; A61K 31/737; A61K 31/167; A61K 31/196; A61K 31/573; A61K 47/02; A61K 9/107

(57) ABSTRACT

The present invention relates to novel medicinal and/or pharmaceutical compositions indicated as composition A and composition B in liquid form for use as a medicinal composition or medicament for intravesical instillation, in the simultaneous local treatment of diseases of the urethra and/or the bladder, where compositions A and B are for use advantageously in the treatment of bladder pain syndrome (interstitial cystitis), in the urethra, and by replenishment of the GAG-layer on the inner surface of the bladder, further advantageously composition A is for use for local analgesic and anaesthetic treatment of the urethra and/or the bladder, and further advantageously the treatment of inflammation of the urethra and/or the bladder and preparation thereof. According to the use of the compositions of the subject matter of the invention, the treatment of the IC/BPS is implemented in two steps using first composition A and secondly composition B for intravesical instillation through the urethra. Osmolarity and pH of the compositions are also optimized. According to the subject matter of the invention composition A comprises the following components: Local anaesthetic, advantageously Lidocaine or adequate salt thereof, corticosteroid, advantageously dexamethasone-disodium-diphosphate or non-steroid anti-inflammatory agent, advantageously diclofenac salt, where furthermore advantageously the local anaesthetic and the non-steroid anti-inflammatory agent and advantageous forms thereof are embedded in liposome or further advantageously are composing complex with a complex composing agent; alkaline basic, advantageously sodium hydroxide; sterile distilled water; alkaline salt, advantageously sodium chloride. According to the subject matter of the invention, furthermore, composition B comprises the following components: Hyaluronic acid or adequate alkaline salt thereof, advantageously sodium-hyaluronate, alkaline salt of chondroitin sulfate, advantageously sodium-chondroitin-sulfate, heparin, advantageously sodium salt of heparin; alkaline basic, advantageously sodium hydroxide or sodium hydrogen carbonate, sterile distilled water, alkaline salt, advantageously sodium chloride and alkaline earth metal salt, advantageously calcium chloride.

The present invention relates to novel medicinal and/or pharmaceutical compositions indicated as composition A and composition B in liquid form for use as a medicinal composition or medicament for intravesical instillation, in the local treatment of diseases of the urethra and/or the bladder, where compositions A and B are for use advantageously in the treatment of bladder pain syndrome (interstitial cystitis) in the urethra, and by replenishment of the GAG-layer on the inner surface of the bladder, further advantageously composition A is for use for local anaesthetic and/or analgesic treatment of the urethra and/or the bladder, and further advantageously the treatment of inflammation of the urethra and/or the bladder.

According to the subject matter of the invention composition A comprises the following components all qualified as Ph. Eur pharmaceutical agent for human use:
1. Local anaesthetic, advantageously Lidocaine or adequate salt thereof, especially advantageously Lidocaine hydrochloride; advantageously embedded in liposome of 1 ml oil-in-water type emulsion, further advantageously composing complex with a complex composing agent, especially advantageously with 2-hydroxypropyl-alpha-cyclodextrin or 2-hydroxypropyl-beta-cyclodextrin or 2-hydroxypropyl-gamma-cyclodextrin;
2. Corticosteroid, advantageously dexamethasone-disodium-diphosphate;
3. Alkaline basic, advantageously sodium hydroxide;
4. Sterile distilled water;
5. Alkaline salt, advantageously sodium chloride.

As a further solution according to the subject matter of the invention composition A comprises the following components all qualified as Ph. Eur pharmaceutical agent for human use:
1. Non-steroid anti-inflammatory agent, advantageously diclofenac, more advantageously diclofenac salt, especially advantageously diclofenac sodium where furthermore advantageously the non-steroid anti-inflammatory agent and advantageous forms thereof are embedded in liposome or further advantageously are composing complex with a complex composing agent, especially advantageously with 2-hydroxypropyl-alpha-cyclodextrin or 2-hydroxypropyl-beta-cyclodextrin or 2-hydroxypropyl-gamma-cyclodextrin;
2. Sterile distilled water;
3. Alkaline salt, advantageously sodium chloride.

As a further solution according to the subject matter of the invention composition A comprises the following components all qualified as Ph. Eur pharmaceutical agent for human use:
1. Local anaesthetic, advantageously Lidocaine or adequate salt thereof, especially advantageously Lidocaine hydrochloride; advantageously embedded in liposome of 1 ml oil-in-water type emulsion; further advantageously composing complex with a complex composing agent, especially advantageously with 2-hydroxypropyl-alpha-cyclodextrin or 2-hydroxypropyl-beta-cyclodextrin or 2-hydroxypropyl-gamma-cyclodextrin;
2. Non-steroid anti-inflammatory agent, advantageously diclofenac, more advantageously diclofenac salt, especially advantageously sodium diclofenac where furthermore advantageously the non-steroid anti-inflammatory agent and advantageous forms thereof are embedded in liposome or further advantageously is composing complex with a complex composing agent, advantageously with 2-hydroxypropyl-alpha-cyclodextrin or 2-hydroxypropyl-beta-cyclodextrin or 2-hydroxypropyl-gamma-cyclodextrin.
3. Alkaline basic, advantageously sodium hydroxid;
4. Sterile distilled water;
5. Alkaline salt, advantageously sodium chloride.

The present invention relates to the following novel and optimal consistence of the composition A in 15 ml (in case of advantageous values) of solution of the composition:
0.25 g-0.6 g, advantageously 0.3 g Lidocaine hydrochloride; advantageously embedded in liposome of 1 ml oil-in-water type emulsion or composing complex with a complex composing agent, especially advantageously with 2-hydroxypropyl-alpha-cyclodextrin or 2-hydroxypropyl-beta-cyclodextrin or 2-hydroxypropyl-gamma-cyclodextrin;
4 mg/l ml-12 mg/3 ml advantageously 8 mg/2 ml dexamethasone-disodium-diphosphate sterile water-solution;
1000-1800 μl advantageously 1300 μl 0.5% sterile sodium-hydroxide solution
11.70 ml sterile distilled water;
20-60 mg, advantageously 40 mg sodium chloride.

Furthermore, the subject matter of the invention relates to the above-described medicinal and/or pharmaceutical composition A, where the pH value thereof is between 6.3 and 8.3 advantageously 7.36, which advantageous value is within the normal range of the pH of the blood and therefore the most optimal value for a local treatment of the urethra and the bladder.

Furthermore, the subject matter of the invention relates to the above-described medicinal and/or pharmaceutical composition A, where the value of osmolarity is between 280 and 310 mOsm/l, advantageously 296 mOsm/l, which advantageous value is within the normal range of osmolarity of the blood, and therefore the most optimal value for a local treatment of the urethra and the bladder.

The present invention furthermore relates to the following novel and optimal consistence of the composition A in 11 ml (in case of advantageous values) of solution of the composition:
50 mg-90 mg, advantageously 75 mg sodium diclofenac advantageously embedded in liposome of 1 ml oil-in-water type emulsion or further advantageously composing complex with composing agent especially advantageously with 2-hydroxypropyl-alpha-cyclodextrin or 2-hydroxypropyl-beta-cyclodextrin or 2-hydroxypropyl-gamma-cyclodextrin in 1:1 to 1:4 weight ratio, advantageously 1:2 weight ratio;
10.00 ml sterile distilled water;
60-100 mg. advantageously 80 mg sodium chloride

Furthermore, the subject matter of the invention relates to the above-described pharmaceutical composition A, where the pH value thereof is between 6.3 and 8.3 advantageously 7.14, which advantageous value is within the normal range of the pH of the blood and therefore the most optimal value for a local treatment of the urethra and the bladder.

Furthermore, the subject matter of the invention relates to the above-described pharmaceutical composition A, where the value of osmolarity is between 280 and 310 mOsm/l, advantageously 291 mOsm/l, which advantageous value is within the normal range of osmolarity of the blood, and therefore the most optimal value for a local treatment of the urethra and the bladder.

The present invention furthermore relates to the following novel and optimal consistence of the composition A in 15 ml (in case of advantageous values) of solution of the composition:
0.25 g-0.6 g. advantageously 0.3 g Lidocaine hydrochloride; advantageously embedded in liposome of 1 ml oil-in-water type emulsion, further advantageously composing complex with a complex composing agent, especially advantageously with 2-hydroxypropyl-alpha-cyclodextrin or 2-hydroxypropyl-beta-cyclodextrin or 2-hydroxypropyl-gamma-cyclodextrin;
50 mg-90 mg. advantageously 75 mg sodium diclofenac advantageously embedded in liposome of 1 ml oil-in-water type emulsion or further advantageously composing complex with composing agent especially advantageously with 2-hydroxypropyl-alpha-cyclodextrin or 2-hydroxypropyl-beta-cyclodextrin or 2-hydroxypropyl-gamma-cyclodextrin in 1:1 to 1:4 weight ratio, advantageously 1:2 weight ratio;
1000-1800 μl advantageously 1300 μl 0.5% sterile sodium-hydroxide solution;
10.00 ml sterile distilled water
60-100 mg, advantageously 80 mg sodium chloride

Furthermore, the subject matter of the invention relates to the above-described pharmaceutical composition A, where the pH value thereof is between 6.3 and 8.3 advantageously 7.36, which advantageous value is within the normal range of the pH of the blood and therefore the most optimal value for a local treatment of the urethra and the bladder.

Furthermore, the subject matter of the invention relates to the above-described pharmaceutical composition A, where the value of osmolarity is between 280 and 310 mOsm/l, advantageously 296 mOsm/l, which advantageous value is within the normal range of osmolarity of the blood, and therefore the most optimal value for a local treatment of the urethra and the bladder.

According to the subject matter of the invention, furthermore, composition B comprises the following components, all qualified as Ph. Eur pharmaceutical agent for human use:
1. Hyaluronic acid or adequate alkaline salt thereof, advantageously sodium-hyaluronate;
2. Alkaline salt of chondroitin sulfate, advantageously sodium-chondroitin-sulfate;
3. Heparin, advantageously sodium salt of heparin;
4. Alkaline basic, advantageously sodium hydroxide, further advantageously sodium hydrogen carbonate;
5. Sterile distilled water;
6. Alkaline salt, advantageously sodium chloride and/or alkaline earth metal salt. advantageously calcium chloride.

The present invention relates to the following novel and optimal consistency of the composition B in 19.4 ml (in case of advantageous values) of the solution of the composition:
6-18 ml, advantageously 10 ml of sterile water solution comprising 1.6 advantageously 160 mg sodium-hyaluronate and 2%, advantageously 200 mg sodium chondroitin-sulfate;
1.00 ml-3.13 ml comprising 5000 IU-15650 IU (appr. 31.2-97.65 mg), advantageously 1.25 ml of sterile medicament 25 000 IU Heparibene Na comprising 6250 IU (appr. 39 mg) of the sodium salt of heparin;
130-190 μl, advantageously 150 μl of 0.5% sterile sodium hydroxide solution or sodium hydrogen carbonate solution;
8 ml of sterile water;
81.5-90 mg, advantageously 85.4 mg sodium chloride and/or
83-92 mg, advantageously 87 mg calcium chloride

Furthermore, the subject matter of the invention relates to the above-described pharmaceutical composition B where the pH value thereof is between 6.3 and 8.3 advantageously 7.38 which advantageous value is within the normal range of the pH of the blood and therefore the most optimal value for local treatment of the urethra and the bladder.

The subject matter of the invention furthermore relates to the process for the preparation of medicinal and/or pharmaceutical composition A by formulating to a medicinal and/or pharmaceutical composition in liquid form by the following steps by using sterile devices:
0.30 g Lidocaine hydrochloride or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio was dissolved in 11.7 ml sterile distilled water and afterword 40 mg of sodium chloride was added and dissolved in the solution.
After adding 8 mg/2 ml dexamethasone-disodium-diphosphate sterile water solution and afterword the 1300 μl 0.5% sterile sodium hydroxide solution, the solution was filtered to sterile on a Sartorius membrane filter with 0.2 μm diameter by vacuum.

The subject matter of the invention furthermore relates to the process for the preparation of medicinal and/or pharmaceutical composition A by formulating to a medicinal and/or pharmaceutical composition in liquid form by the following steps by using sterile devices:
40 mg sodium chloride was dissolved in 11.7 ml sterile distilled water and after adding the 8 mg/2 ml dexamethasone-disodium-diphosphate sterile water solution and afterword the 1300 μl 0.5% sterile sodium hydroxide solution, the solution was filtered to sterile on a Sartorius membrane filter with 0.2 μm diameter by vacuum. After adding 0.30 g liposomal Lidocaine hydrochloride in 1 ml of oil-in-water type emulsion to the resulted sterile solution the alloy was homogenized.

For calculation of the proper sodium-hydroxide quantity, the value of pH was measured by Jenway 3510 pH Meter.

Calibration of the device was made by puffer solutions on two points with pH values 4.01 and 7.00.

The subject matter of the invention furthermore relates to the process for the preparation of medicinal and/or pharmaceutical composition A by formulating to a medicinal and/or pharmaceutical composition in liquid form by the following steps by using sterile devices:
80 mg sodium chloride was dissolved in 10 ml sterile distilled water, and afterword 75 mg diclofenac or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio was added and dissolved in the solution.
Afterword the solution was filtered to sterile on a Sartorius membrane filter with 0.2 μm diameter by vacuum.

The subject matter of the invention furthermore relates to the process for the preparation of medicinal and/or pharmaceutical composition A by formulating to a medicinal and/or pharmaceutical composition in liquid form by the following steps by using sterile devices:
80 mg sodium chloride was dissolved in 10 ml sterile distilled water and afterword the solution was filtered to sterile on a Sartorius membrane filter with 0.2 μm diameter by vacuum. After adding 75 mg liposomal diclofenac in 1 ml of oil-in-water type emulsion to the resulted sterile sodium chloride solution the alloy was homogenized.

The value of pH was measured by Jenway 3510 pH Meter.

Calibration of the device was made by puffer solutions on two points with pH values 4.01 and 7.00.

The subject matter of the invention furthermore relates to the process for the preparation of medicinal and/or pharmaceutical composition A by formulating to a medicinal and/or pharmaceutical composition in liquid form by the following steps by using sterile devices:
40 mg sodium chloride was dissolved in 10 ml sterile distilled water, and afterword 0.3 g Lidocaine hydrochloride or the complex thereof and 75 mg diclofenac or the complex thereof both complexes formed with 2-hydroxypropyl-alpha-cyclodextrin or 2-hydroxypropyl-beta-cyclodextrin or 2-hydroxypropyl-gamma-cyclodextrin in 1:2 weight ratio was added and dissolved in the solution.
After adding 1300 μl 0.5% sterile sodium hydroxide solution, the solution was filtered to sterile on a Sartorius membrane filter with 0.2 μm diameter by vacuum.

The subject matter of the invention furthermore relates to the process for the preparation of medicinal and/or pharmaceutical composition A by formulating to a medicinal and/or pharmaceutical composition in liquid form by the following steps by using sterile devices:
40 mg sodium chloride was dissolved in 10 ml sterile distilled water and after adding 1300 μl 0.5% sterile sodium hydroxide solution the solution was filtered to sterile on a Sartorius membrane filter with 0.2 μm diameter by vacuum. After adding 0.3 g liposomal Lidocaine hydrochloride in 1 ml oil-in-water type emulsion and 75 mg liposomal diclofenac in 1 ml of oil-in-water type emulsion to the resulted sterile solution by filtering the alloy was homogenized.

The subject matter of the invention furthermore relates to the process for the preparation of medicinal and/or pharmaceutical composition A by formulating to a medicinal and/or pharmaceutical composition in liquid form by the following steps by using sterile devices:
40 mg sodium chloride was dissolved in 10 ml sterile distilled water and after adding and dissolving 75 mg diclofenac or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio and 1300 μl 0.5% sterile sodium hydroxide solution the solution was filtered to sterile on a Sartorius membrane filter with 0.2 μm diameter by vacuum.
After adding 0.3 g liposomal Lidocaine hydrochloride in 1 ml oil-in-water type emulsion to the resulted sterile solution by filtering the alloy was homogenized.

The subject matter of the invention furthermore relates to the process for the preparation of medicinal and/or pharmaceutical composition A by formulating to a medicinal and/or pharmaceutical composition in liquid form by the following steps by using sterile devices:
40 mg sodium chloride was dissolved in 10 ml sterile distilled water, and afterword 0.3 g Lidocaine hydrochloride or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio was added and dissolved in the solution. After adding 1300 μl 0.5% sterile sodium hydroxide solution, the solution was filtered to sterile on a Sartorius membrane filter with 0.2 μm diameter by vacuum.
After adding 75 mg liposomal diclofenac in 1 ml of oil-in-water type emulsion to the resulted sterile solution by filtering the alloy was homogenized.

For calculation of the proper sodium-hydroxide quantity the value of pH was measured by Jenway 3510 pH Meter.

Calibration of the device was made by puffer solutions on two points with pH values 4.01 and 7.00.

For setting the proper osmolarity of composition A and B and for calculation of the proper sodium chloride quantity, the value of osmolarity was measured by Gonotec type Osmomat 3000 point of congelation osmometer.

Calibration of the device was made on two points by distilled water on value 0 mOsm/l and by a standard calibration solution (NaCl/H₂O) on value 300 mOsm/l.

The sterile solution or emulsion of composition A was presented in a polypropylene syringe produced by Becton Dickinson.

All steps of preparation were made in a laminar cabin with horizontal air-flow

The subject matter of the invention furthermore relates to the process for the preparation of medicinal and/or pharmaceutical composition B by formulating to a medicinal and/or pharmaceutical composition in liquid form by the following steps:
After mixing the 150 μl 0.5% sterile sodium hydroxide or sodium hydrogen carbonate solution with 8 ml sterile water and dissolving the 85.4 mg sodium chloride, the solution was filtered to sterile on a Sartorius membrane filter with 0.2 μm diameter by vacuum and was mixed afterword with 10 ml of sterile water solution comprising 160 mg sodium-hyaluronate (1.6%), 200 mg sodium-chondroitin-sulfate (2%), 87 mg calcium chloride and 1.25 ml of medicament 25 000 IU Heparibene Na, comprising 5250 IU (appr. 39 mg) of sodium salt of heparin
For calculation of the proper sodium-hydroxide or sodium hydrogen carbonate quantity, the value of pH was measured by Jenway 3510 pH Meter.

Calibration of the device was made by puffer solutions on two points with pH values 4.01 and 7.00.

For setting the proper osmolarity and for calculation of the proper sodium chloride quantity, the value of osmolarity was measured by Gonotec type Osmomat 3000 point of congelation osmometer.

Calibration of the device was made on two points by distilled water on value 0 mOsm/l and by a standard calibration solution (NaCl/H₂O) on value 300 mOsm/l.

The sterile solution of composition B was presented in a polypropylene syringe produced by Becton Dickinson.

All steps of preparation were made in a laminar cabin with horizontal air-flow.

The subject matter of the invention furthermore relates to medicinal and/or pharmaceutical compositions A and B for use in the treatment for bladder pain syndrome (interstitial cystitis) in two steps, first by using the composition A for intravesical instillation through the urethra and afterword secondly 2-8 minutes, advantageously 4 minutes later by using composition B for intravesical instillation through the urethra.

The subject matter of the invention furthermore relates to the compositions A and B for use in the treatments described above, where compositions can be administered by intravesical instillation through the urethra using a catheter or by a catheter- and pain-free instillation using a urological syringe adapter also innovated by Dr. Lovász et al.

Using the innovated urological syringe adapter, the local treatment of the urethra is also possible by the compositions according to the invention.

### HISTORY, THE STATE OF THE ART

The prior art referred and cited in the present specification from now on are all part of state of the art.

Interstitial Cystitis or Bladder Pain Syndrome (IC/BPS) is a lesser-known disease. However, its symptoms can be severe, and there is no known cure for it. Presently its diagnosis rate is low, and it is often being mistreated, which makes the symptoms even worse.

IC/BPS is prevalent all around the world. It is a bladder disease of unknown etiology. The typical symptoms are bladder and pelvic pain or discomfort, urinary urgency, and frequency. All of these can have a detrimental effect on the patient's quality of life, by obstructing working abilities, sexual intercourse, sleep, and many other activities.

Not only can it be hard to diagnose IC/BPS, but also there is no known treatment that can cure the disease forever. Although in many countries (including the ones with the most advanced health-care) there is no effective way to cure IC/BPS, by using the proper method it could be made symptomless. For achieving this condition, regular treatments and a constant—in many cases life-long—follow-up is needed.

In most countries, IC/BPS is usually treated with oral medicines. The efficacy of these oral compositions are low, and also side-effects are more frequent. Local treatment (bladder instillation) should be the best option, but there is neither medicine nor medicinal composition of good efficacy yet. Moreover, instillation is performed through a catheter, which is painful in many cases and it can cause hemorrhagic lesions, too.

The inner surface of the bladder mucosa is covered by a mucous layer.

The mucosa of the bladder consists of a multi-layered transitional epithelium (urothelium) with a special glycosaminoglycan (GAG) layer, which enables the storage of the urine with a high osmotic gradient to the blood.

If this layer becomes damaged, the components of the urine cause a chronic chemical irritation of the deeper layers of the bladder wall. The condition progresses into a non-bacterial inflammation, which causes pain of different intensity, abnormal voiding frequency, and/or urgency. All of these symptoms adversely influence the patients' sleeping, working, sexual, social, and exercise activities. Without proper treatment, the disease progresses and leads to a chronic bladder or kidney failure. These conditions are irreversible.

IC/BPS can show up in all age groups, both genders, and in all races. It is 5-10 times more common in women than in men, though. Due to the low diagnosis rate, it is hard to assess the prevalence of IC/BPS. The only assumptions we can make are based on data from the USA, Hungary, and certain other countries. According to most estimations, the prevalence of IC/BPS is between 200-400 persons per 100.000 people (which means a rate of 0.2-2%). That said, in Hungary, there have to be at least 20,000-40,000 people who are affected. The diagnosed cases are merely 500-600.

This means a rate of 2-3% or less, which is abysmal, even if the rate in countries with more advanced health-care tops currently at about 10%.

The etiology of IC/BPS is still not known. It is proven, on the other hand, that the symptoms develop due to the insufficiency of the GAG-layer which covers the inner, mucosal surface of the bladder. The main role of the GAG-layer is to protect the deeper layer of the bladder wall from the irritative solutions of the urine.

In case of IC/BPS the GAG-layer becomes permeable to the soluble components of the urine and with time a chronic, sterile inflammation develops (which is not caused by bacteria) in the deeper layers of the bladder wall. This leads to severe pain.

Most urologists throughout the world are focusing primarily on oncological, prostatic, and erection problems. Therefore just a few of them have appropriate knowledge of IC/BPS. With time the patient's quality of life is getting worse and worse: the permanent urgency of voiding and the severe pain have a detrimental effect on everyday activities, too.

IC/BPS is the disease which one of the inventors, Sandor Lovasz MD. PhD., urologist, therapist, and his co-workers started to focus on 10 years ago. While diagnosing and treating several patients, they started to ponder how the treatment can be made better and less painful by developing new, innovative devices.

Partly because of these other innovations (urological syringe adapter and assisting device for self-instillation) the interest of the local treatment of the IC/BPS increased a lot during the past years.

The most important mode of IC/BPS therapy is the GAG-layer replenishment.

GAG-layer replenishment is a cornerstone in the therapy of IC/BPS. During the last years, intravesical GAG layer replenishment has proven to be the most efficacious treatment also for overactive bladder (OAB), radiation cystitis, and recurrent urinary tract infections (UTIs).

The pharmaceutical industry has been trying to find a viable method for this problem for more than 50 years, but with little effect so far. The only medicine in the USA approved by FDA is Elmiron, which is an oral medicine with an active agent of polysaccharide called pentosan polysulfate sodium (PPS). It was approved by FDA 35 years ago. The main drawback of this oral medicine is that merely the 5% of the active agent gets absorbed, what considerably lowers its efficacy. So far, there has been no other agent used for direct bladder treatment, which resulted in a significant improvement of the symptoms in a clinical trial. Recently there have been scientific papers published about the side effects of taking PPS over a long time. Among these, the most distressing one is pigmentary maculopathy, which is a severe visual disorder.

This new information will make the market even emptier than it has been—and the need for a remedy of scientifically proven efficacy will be even higher, especially because for most of the patients the only therapy which brings relief is bladder instillation.

Our solution are the invention of two special, multi-component cocktails, medicinal and/or pharmaceutical compositions of unique specifications developed by the inventors for the local treatment of IC/BPS by the replenishment of the GAG-layer of the bladder, including an introductory anaesthetic and/or anti-inflammatory treatment of the urethra and/or the bladder which is part of the treatment of IC/BPS in the urethra and the bladder.

According to the prior art local treatment of the bladder by liposomal agents is well known but using liposomal agent as an introductory treatment before the GAG replenishment is a novel procedure.

Therefore the use of the anti-inflammatory agent of the first cocktail A embedded in liposome according to the subject matter of the invention is a very effective way to treat the IC/BPS in bladder.

As a further solution using complex composing agents advantageously 2-hydroxypropyl-alpha-cyclodextrin or further advantageously 2-hydroxypropyl-beta-cyclodextrin or further advantageously 2-hydroxypropyl-gamma-cyclodextrin in the composition A keeping the solution of composition A stable through composing a complex with lidocaine or diclofenac or with any of the salts thereof according to the subject matter of the invention is a very effective way to treat the IC/BPS in bladder as well.

Using the liposomal or complex forms of the agents of composition A helps in the absorption and the inhibition of any aggregation of the active agents.

Because Sandor Lovasz MD., one of the inventors has been treating about 540 patients on his own and the number of his patients is increasing by about 100 in every year, the sheer number of patients proves the efficacy of the compositions he uses.

### SUMMARY

The subject matter of the invention are two special, multi-component cocktails, medicinal and/or pharmaceutical compositions (indicated as A and B compositions) of unique specifications developed by the inventors for the local treatment of IC/BPS in the urethra and/or by replenishment of the GAG-layer in the bladder, including an introductory local anaesthetic and/or analgesic, anti-inflammatory treatment of the urethra and/or the bladder, which is part of the simultaneous treatment of the urethra and the bladder in IC/BPS.

The reasons why all the compositions of the state of art conventionally used for local treatment of the IC/BPS are less efficacious then the compositions of the subject matter of the invention are the following:
1) According to the use of the compositions of the subject matter of the invention. the treatment of the IC/BPS is implemented in two steps using first composition A and secondly 2-8 minutes advantageously 4 minutes later composition B for intravesical instillation through the urethra.
   The two-step treatment is important,
   a) because using the compositions at once, the composition used for GAG layer replenishment (composition B) hinders the efficacy of the components composition A used for the treatment of inflammatory (steroid or nonsteroid anti-inflammatory agent advantageously) and local anaesthetic (Lidocaine hydrochloride) or analgesic (diclofenac or liposomal sodium diclofenac complex of sodium diclofenac) instillation by creating an immediate artificial layer on the inner surface of the bladder.
   b) Moreover if the local anaesthetic agent (Lidocaine hydrochloride) and corticosteroid and/or the liposomal diclofenac would be given together in one cocktail with the agents for GAG replenishment (which is the solution of state of the art) efficacy will be lost because of the dilution thereof.
   c) Moreover the liposomal or the complex forms of the instilling agents of composition A are advantageous because the absorption of the active agents in the bladder is provided this way and the aggregation of the agents in composition A is inhibited specially in case components of composition B is mixed with those components of composition A in the bladder.
   d) Using composition A as the first instilled agent, and causing a local anaesthetic or analgesic effect is important because this is the reason, why patients can keep the solution B in the bladder for a longer-term (more, than 3 hours).
2) Composition B comprises all the three main compounds of the GAG layer while all the other cocktails of the state of the art conventionally used earlier comprise only one or two compounds thereof.
   Therefore, by using the composition B with all the three GAG-layer compounds, the efficacy of the GAG-layer replenishment can significantly be improved.
3) The pH values of all previously used solutions proved to be too much acidic and therefore irritating. This is the reason why the pH value of the compositions is optimized which is part of the subject matter of the invention as well.
4) The value of osmolarity of the compositions is also important for reducing the irritating effect of the composition which is part of the subject matter of the invention as well.

### REFERENCES

Recent Advances in Intravesical Drug/Gene Delivery Pradeep Tyagi. Pao-Chu Wu. Michael Chancellor, Naoki Yoshimura, Leaf Huang Mol Pharm. Author manuscript; available in PMC 2008 Aug. 8.
Recent developments of intravesical therapy of painful bladder syndrome/interstitial cystitis: a review Toft, Benedikte Richter; Nordling, Jorgen Current Opinion in Urology Volume 16(4), July 2006, p 268-272
Interstitial cystitis intravesical therapy Tanya Ha, Jie Hua Xu Transl Androl Urol. 2017 July; 6(Suppl 2): S171-S179
State of the Art in Intravesical Therapy for Lower Urinary Tract Symptoms Jonathan Kaufman, Vikas Tyagi, Michele Anthony, Michael B Chancellor, Pradeep Tyagi Rev Urol. 2010 Fall; 12(4): e181-e189.
Combined intravesical sodium hyaluronate/chondroitin sulfate therapy for interstitial cystitis/bladder pain syndrome: a prospective study Claudio Giberti, Fabrizio Gallo, Pierluigi Cortese, Maurizio Schenone Ther Adv Urol. 2013 August; 5(4): 175-179.
Novel targeted bladder drug-delivery systems: a review Martino Maria Zacche, Sushma Srikrishna, Linda Cardozo Res Rep Urol. 2015; 7: 169-178. Published online 2015 Nov. 23.
GAG replenishment therapy for bladder pain syndrome/interstitial cystitis. Wyndaele J J J, Riedl C, Taneja R, Lovisz S, Ueda T, Cervigni M Neurourol Urodyn [28 Dec. 2018, 38(2):535-544]
Intravesical drug delivery: Challenges, current status, opportunities and novel strategies Shruti Guha Sarkar, R. Banerjee Journal of Controlled Release Volume 148, Issue 2, 1 Dec. 2010, Pages 147-159
Novel targeted bladder drug-delivery systems: a review Martino Maria Zacche Sushma Srikrishna Linda Cardozo Department of Urogynaecology, King's College Hospital. London, UK Research and Reports in Urology 2015:7 169-178
Efficacy and Safety Profile of Diclofenac/Cyclodextrin and Progesterone/Cyclodextrin Formulations: A Review of the Literature Data Cristina Scavone et 1 Drugs R D. 2016 June; 16(2): 129-140. Published online 2016 Mar. 3. doi: 10.1007/s40268-016-0123-2

1. Novel medicinal and/or pharmaceutical composition indicated as composition A in liquid form for use as a medicinal composition or medicament for intravesical instillation in the local treatment of diseases of the urethra and/or the bladder
where A composition comprises the following components:
Local anaesthetic agent;
Corticosteroid agent
or
Non-steroid anti-inflammatory agent;
Alkaline basic;
Sterile distilled water;
Alkaline salt.

2. Novel medicinal and/or pharmaceutical composition indicated as composition A in liquid form for use as a medicinal composition or medicament for intravesical instillation in the local treatment of diseases of the urethra and/or the bladder
where A composition comprises the following components:
Non-steroid anti-inflammatory agent;
Sterile distilled water;
Alkaline salt.

3. Novel medicinal and/or pharmaceutical composition indicated as composition B in liquid form for use as a medicinal composition or medicament for intravesical instillation, in the local treatment of diseases of the urethra and/or the bladder
where B composition comprises the following components:
Hyaluronic acid, or an adequate alkaline salt of hyaluronic acid;
Alkaline salt of chondroitin sulfate;
Heparin, or an adequate alkaline salt of heparin;
Alkaline basic;
Sterile distilled water;
Alkaline salt and/or alkaline earth metal salt.

4. Composition A according to any of claims 1 to 2 characterized in that composition A is for use for local anaesthetic and/or analgesic treatment of the urethra and/or the bladder and/or in the local treatment of inflammation and/or of bladder pain syndrome (interstitial cystitis) in the urethra and/or the bladder.

5. Composition B according to claim 3 characterized in that composition B is for use in the local treatment of bladder pain syndrome (interstitial cystitis) in the urethra and/or by GAG layer replenishment in the bladder.

6. Composition A according to any of claims 1 and 4 characterized in that the local anaesthetic agent is Lidocaine or adequate salt thereof, advantageously Lidocaine hydrochloride, the corticosteroid agent is dexamethasone-disodium-diphosphate, the alkaline basic is sodium hydroxide and the alkaline salt is sodium chloride.

7. Composition A according any of claims 1, 4 and 6 characterized in that the novel and optimal consistency of the composition A is as listed as follows:
0.25 g-1.0 g Lidocaine hydrochloride; or liposomal Lidocaine hydrochloride in 1 ml of oil-in-water type emulsion or the complex form of Lidocaine hydrochloride formed with 2-hydroxypropyl-alpha-cyclodextrin or 2-hydroxypropyl-beta-cyclodextrin or 2-hydroxypropyl-gamma-cyclodextrin in 1:1 to 1:4 weight ratio;
4 mg/l ml-12 mg/3 ml dexamethasone-disodium-diphosphate sterile water-solution;
1000-1800 μl sterile sodium-hydroxide solution;
11.70 ml sterile distilled water;
20-60 mg sodium chloride.

8. Composition A according to claim 7 characterized in that the values of the optimal consistence in 15 ml solution of composition A is as listed as follows:
0.30 g Lidocaine hydrochloride; or liposomal Lidocaine hydrochloride in 1 ml of oil-in-water type emulsion or the complex form of Lidocaine hydrochloride formed with 2-hydroxypropyl-alpha-cyclodextrin or 2-hydroxypropyl-beta-cyclodextrin or 2-hydroxypropyl-gamma-cyclodextrin in 1:2 weight ratio;
8 mg/2 ml dexamethasone-disodium-diphosphate sterile water solution;
1 300 μl 0.5% sterile sodium-hydroxide solution;
11.70 ml sterile distilled water;
40 mg of sodium chloride.

9. Composition A according to any of claims 1, 4 and 6 to 8 characterized in that the optimal pH value thereof is between 6.3 and 8.3, and the optimal value of osmolarity is between 280 and 310 mOsm/l.

10. Composition A according to claim 9 characterized in that the optimal pH value thereof is 7.36, and the optimal value of osmolarity is 296 mOsm/l.

11. Composition A according to any of claims 1 and 2 characterised in that the non-steroid anti-inflammatory agent is diclofenac salt.

12. Composition A according to claim 11 characterised in that the non-steroid anti-inflammatory agent is sodium diclofenac.

13. Composition A according to any of claims 1, 2, 6 to 8 and 11 to 12 characterised in that the local anaesthetic and the non-steroid anti-inflammatory agent are embedded in liposome of oil-in water type emulsion.

14. Composition A according to any of claims 1, 2, 6 to 8 and 11 to 12 characterised in that the local anaesthetic and the non-steroid anti-inflammatory agent are a complex formed with 2-hydroxypropyl-alpha-cyclodextrin or 2-hydroxypropyl-beta-cyclodextrin or 2-hydroxypropyl-gamma-cyclodextrin in 1:1 to 1:4 weight ratio.

15. Composition A according to claim 14 characterised in that the weight ratio is 1:2.

16. Composition A according to any of claims 1 to 15 characterised in that the alkaline salt is sodium chloride.

17. Composition A according to any of claims 2 and 11 to 16 characterized in that the optimal pH value thereof is 7.14, and the optimal value of osmolarity is 291 mOsm/l.

18. Composition A according to any of claims 1 and 4 characterized in that the local anaesthetic agent is Lidocaine or adequate salt thereof, advantageously Lidocaine hydrochloride, the non-steroid anti-inflammatory agent is diclofenac salt, advantageously sodium diclofenac, the alkaline basic is sodium hydroxide and the alkaline salt is sodium chloride.

19. Composition A according to any of claims 2 and 4 characterized in that the non-steroid anti-inflammatory agent is diclofenac salt, advantageously sodium diclofenac and the alkaline salt is sodium chloride.

20. Composition A according any of claims 1, 4 and 6 characterized in that the novel and optimal consistency of the composition A is as listed as follows:
0.25 g-1.0 g Lidocaine hydrochloride or liposomal Lidocaine hydrochloride in 1 ml of oil-in-water type emulsion or the complex form of Lidocaine hydrochloride formed with 2-hydroxypropyl-alpha-cyclodextrin or 2-hydroxypropyl-beta-cyclodextrin or 2-hydroxypropyl-gamma-cyclodextrin in 1:1 to 1:4 weight ratio;
50 mg-90 mg liposomal sodium diclofenac in 1 ml of oil-in-water type emulsion or the complex form of sodium diclofenac according to the claims 14 and 15;
1000-1800 μl sterile sodium-hydroxide solution;
11.70 ml sterile distilled water;
20-60 mg sodium chloride.

21. Composition A according to claim 20 characterized in that the values of the optimal consistence in 15 ml solution of composition A is as listed as follows:
0.30 g Lidocaine hydrochloride; or liposomal Lidocaine hydrochloride in 1 ml of oil-in-water type emulsion or the complex form of Lidocaine hydrochloride formed with 2-hydroxypropyl-alpha-cyclodextrin or 2-hydroxypropyl-beta-cyclodextrin or 2-hydroxypropyl-gamma-cyclodextrin in 1:2 weight ratio;
75 mg liposomal sodium diclofenac in 1 ml of oil-in-water type emulsion or the complex form of sodium diclofenac according to the claims 14 and 15
1 300 μl 0.5% sterile sodium-hydroxide solution;
11.70 ml sterile distilled water;
40 mg of sodium chloride.

22. Composition A according to claims 2, 11 to 15, characterized in that the values of the optimal consistence in 11 ml solution composition A is as listed as follows:
50 mg-90 mg sodium diclofenac or liposomal sodium diclofenac in 1 ml of oil-in-water type emulsion or the complex form of sodium diclofenac according to the claims 14 and 15;
10.00 ml sterile distilled water
60-100 mg, advantageously 80 mg sodium chloride.

23. Composition A according to claim 16, characterized in that the values of the optimal consistence in 11 ml solution composition A is as listed as follows:
75 mg sodium diclofenac or liposomal sodium diclofenac in 1 ml of oil-in-water type emulsion or the complex form of sodium diclofenac according to the claims 14 and 15;
10.00 ml sterile distilled water
80 mg sodium chloride.

24. Composition B according to any of claims 3 and 5 characterized in that the alkaline salt of hyaluronic acid is sodium-hyaluronate; the alkaline salt of chondroitin sulfate is sodium-chondroitin-sulfate; the used form of heparin is medicament 25000 IU Heparibene Na comprising sodium salt of heparin, the alkaline basic is sodium hydroxide or sodium hydrogen carbonate, the alkaline salt is sodium chloride, and the alkaline earth metal salt is calcium chloride.

25. Composition B according to any of claims 3, 5 and 24 characterized in that the novel and optimal consistency of the composition B is as listed as follows:
6-18 ml sterile water solution comprising 1.6% of sodium-hyaluronate, 2% of sodium-chondroitin sulfate and 0.87% of calcium chloride;
1.00 ml-3.13 ml advantageously 1.25 ml of sterile medicament 25 000 IU Heparibene Na comprising 5000 IU-15650 IU (appr. 31.2-97.65 mg) of the sodium salt of heparin;
130-190 μl of 0.5% sterile sodium hydroxide or sodium hydrogen carbonate solution;
8 ml of sterile water;
81.5-90 mg sodium chloride.

26. Composition B, according to claim 25 characterized in that the values of the optimal consistence in 19.4 ml solution is as listed as follows:
10 ml of sterile water solution comprising 160 mg sodium-hyaluronate (1.6%), 200 mg sodium-chondroitin-sulfate (2%) and 87 mg calcium chloride (0.87%);
1.25 ml of sterile medicament 25 000 IU Heparibene Na comprising 6250 IU (appr. 39 mg) sodium salt of heparin;
150 μl 0.5% sterile sodium hydroxide or sodium hydrogen carbonate solution;
8 ml sterile water;
85.4 mg sodium chloride.

27. Composition B according to any of claims 3, 5 and 24 to 26 characterized in that the optimal pH value thereof is between 6.3 and 8.3, and the optimal value of osmolarity is between 280 and 310 mOsm/l.

28. Composition B according to claim 27 characterized in that the optimal pH value thereof is 7.38, and the optimal value of osmolarity is 299 mOsm/l.

29. Medicinal and/or pharmaceutical compositions A and B according to any of claims 1 to 28 for use in treatment for bladder pain syndrome (interstitial cystitis) in two steps, first by using the composition A for intravesical instillation and afterword secondly 2-8 minutes later by using composition B for intravesical instillation through the urethra.

30. Medicinal and/or pharmaceutical compositions A and B according to claim 29 characterized in that the use of composition B for intravesical instillation comes 4 minutes later after the intravesical instillation of composition A.

31. Medicinal and/or pharmaceutical compositions A and B according to any of claims 1 to 28 for use in the treatments according to any of claims 1 to 6 and 29 to 30 characterized in that compositions can be administered by intravesical instillation through the urethra using a catheter treating only the bladder or by a catheter- and pain-free instillation using a urological syringe adapter treating simultaneously the urethra and the bladder.

32. Process for the preparation of medicinal and/or pharmaceutical composition A according to any of claims 1, 4 and 6 to 10 and 18 by formulating to a medicinal and/or pharmaceutical composition in liquid form by the following steps by using sterile devices:
0.30 g Lidocaine hydrochloride or a complex thereof formed with 2-hydroxypropyl-alpha-βcyclodextrin or 2-hydroxypropyl-beta-βcyclodextrin or 2-hydroxypropyl-gamma-βcyclodextrin in 1:2 weight ratio was dissolved in 11.7 ml sterile distilled water and afterword 40 mg of sodium chloride was added and dissolved in the solution.
After adding 8 mg/2 ml dexamethasone-disodium-diphosphate sterile water solution and afterword the 1300 μl 0.5% sterile sodium hydroxide solution, the solution was filtered to sterile on a Sartorius membrane filter with 0.2 μm diameter by vacuum.

33. Process for the preparation of medicinal and/or pharmaceutical composition A according to any of claims 1, 4 and 6 to 10 and 18 by formulating to a medicinal and/or pharmaceutical composition in liquid form by the following steps by using sterile devices:
40 mg sodium chloride was dissolved in 11.7 ml sterile distilled water and after adding the 8 mg/2 ml dexamethasone-disodium-diphosphate sterile water solution and afterword the 1300 μl 0.5% sterile sodium hydroxide solution, the solution was filtered to sterile on a Sartorius membrane filter with 0.2 μm diameter by vacuum. After adding 0.30 g liposomal Lidocaine hydrochloride in 1 ml of oil-in-water type emulsion to the resulted sterile solution the alloy was homogenized.

34. Process for the preparation of medicinal and/or pharmaceutical composition A according to any of claims 2, 4 and 11 to 17 and 19 by formulating to a medicinal and/or pharmaceutical composition in liquid form by the following steps by using sterile devices:
80 mg sodium chloride was dissolved in 10 nil sterile distilled water, and afterword 75 mg diclofenac or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio was added and dissolved in the solution.
Afterword the solution was filtered to sterile on a Sartorius membrane filter with 0.2 μm diameter by vacuum.

35. Process for the preparation of medicinal and/or pharmaceutical composition A according to any of claims 2, 4 and 11 to 17 and 19 by formulating to a medicinal and/or pharmaceutical composition in liquid form by the following steps by using sterile devices:
80 mg sodium chloride was dissolved in 10 ml sterile distilled water and afterword the solution was filtered to sterile on a Sartorius membrane filter with 0.2 μm diameter by vacuum. After adding 75 mg liposomal diclofenac in 1 ml of oil-in-water type emulsion to the resulted sterile sodium chloride solution the alloy was homogenized.

36. Process for the preparation of medicinal and/or pharmaceutical composition A according to any of claims 1, 4 and 6 to 10 and 18 to 19 by formulating to a medicinal and/or pharmaceutical composition in liquid form by the following steps by using sterile devices:
40 mg sodium chloride was dissolved in 10 ml sterile distilled water, and afterword 0.3 g Lidocaine hydrochloride or a complex thereof and 75 mg diclofenac or a complex thereof both complexes formed with 2-hydroxypropyl-alpha-cyclodextrin or 2-hydroxypropyl-beta-cyclodextrin or 2-hydroxypropyl-gamma-cyclodextrin in 1:2 weight ratio was added and dissolved in the solution.
After adding 1300 μl 0.5% sterile sodium hydroxide solution, the solution was filtered to sterile on a Sartorius membrane filter with 0.2 μm diameter by vacuum.

37. Process for the preparation of medicinal and/or pharmaceutical composition A according to any of claims 1, 4 and 6 to 10 and 18 to 19 by formulating to a medicinal and/or pharmaceutical composition in liquid form by the following steps by using sterile devices:
40 mg sodium chloride was dissolved in 10 ml sterile distilled water and after adding 1300 μl 0.5% sterile sodium hydroxide solution the solution was filtered to sterile on a Sartorius membrane filter with 0.2 μm diameter by vacuum. After adding 0.3 g liposomal Lidocaine hydrochloride in 1 ml oil-in-water type emulsion and 75 mg liposomal diclofenac in 1 ml of oil-in-water type emulsion to the resulted sterile solution by filtering the alloy was homogenized.

38. Process for the preparation of medicinal and/or pharmaceutical composition A according to any of claims 1, 4 and 6 to 10 and 18 to 19 by formulating to a medicinal and/or pharmaceutical composition in liquid form by the following steps by using sterile devices:
40 mg sodium chloride was dissolved in 10 ml sterile distilled water, and afterword 0.3 g Lidocaine hydrochloride or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio was added and dissolved in the solution. After adding 1300 μl 0.5% sterile sodium hydroxide solution, the solution was filtered to sterile on a Sartorius membrane filter with 0.2 μm diameter by vacuum.
After adding 75 mg liposomal diclofenac in 1 ml of oil-in-water type emulsion to the resulted sterile solution by filtering the alloy was homogenized.

39. Process for the preparation of medicinal and/or pharmaceutical composition A according to any of claims 1, 4 and 6 to 10 and 18 to 19 by formulating to a medicinal and/or pharmaceutical composition in liquid form by the following steps by using sterile devices:
40 mg sodium chloride was dissolved in 10 ml sterile distilled water and after adding and dissolving 75 mg diclofenac or the complex thereof formed with 2-hydroxypropyl-alpha-cyclodextrin or 2-hydroxypropyl-beta-cyclodextrin or 2-hydroxypropyl-gamma-cyclodextrin in 1:2 weight ratio and 1300 μl 0.5% sterile sodium hydroxide solution the solution was filtered to sterile on a Sartorius membrane filter with 0.2 μm diameter by vacuum.
After adding 0.3 g liposomal Lidocaine hydrochloride in 1 ml oil-in-water type emulsion to the resulted sterile solution by filtering the alloy was homogenized.

40. Process for the preparation of medicinal and/or pharmaceutical composition B according to any of claims 3, 5 and 24 to 28 by formulating to a medicinal and/or pharmaceutical composition in liquid form by the following steps:
After mixing the 150 μl 0.5% sterile sodium hydroxide solution with 8 ml sterile water and dissolving the 85.4 mg sodium chloride, the solution was filtered to sterile on a Sartorius membrane filter with 0.2 μm diameter by vacuum and was mixed afterword with 10 ml of sterile water solution comprising 160 mg sodium-hyaluronate (1.6%), 200 mg sodium-chondroitin-sulfate (2%), 87 mg calcium chloride and 1.25 ml of medicament 25 000 IU Heparibene Na, comprising 5250 IU (appr. 39 mg) of sodium salt of heparin.

41. Process for the preparation of medicinal and/or pharmaceutical composition A and B according to any of claims 32 to 33 and 36 to 40 characterised in that for calculation of the proper sodium-hydroxide quantity, the optimal value of pH was measured by Jenway 3510 pH Meter device.

42. Process for the preparation of medicinal and/or pharmaceutical composition A according to any of claims 34 to 35 characterised in that the optimal value of pH was measured by Jenway 3510 pH Meter device.

43. Process for the preparation of medicinal and/or pharmaceutical composition A and B according to any of claims 32 to 40 characterised in that
the calibration of the Jenway 3510 pH Meter device was made by puffer solutions on two points with pH values 4.01 and 7.00 and
for setting the proper osmolarity and for calculation of the proper sodium chloride quantity, the value of osmolarity was measured by Gonotec type Osmomat 3000 point of congelation osmometer and
for calibration of the device was made on two points by distilled water on value 0 mOsm/l and by a calibration standard solution (NaCl/H2O) on value 300 mOsm/l. and
the sterile solution of composition A was presented in a polypropylene syringe produced by Becton Dickinson and
all steps of preparation were made in a laminar cabin with horizontal air-flow.
